(19) United States
(12) Patent Application Publication
(10) Pub. No.: US 2022/0221467 A1
(43) Pub. Date: Jul. 14, 2022

(54) SYSTEMS AND METHODS FOR MS1-BASED MASS IDENTIFICATION INCLUDING SUPER-RESOLUTION TECHNIQUES

(71) Applicant: President and Fellows of Harvard College, Cambridge, MA (US)

(72) Inventors: Marc W. Kirschner, Cambridge, MA (US); Mingjie Dai, Cambridge, MA (US); Matthew Sonnett, Cambridge, MA (US); Leonid Peshkin, Cambridge, MA (US)

(73) Assignee: President and Fellows of Harvard College, Cambridge, MA (US)

(21) Appl. No.: 17/613,466

(22) PCT Filed: May 29, 2020
(86) PCT No.: PCT/US2020/035421

§ 371 Date: Nov. 22, 2021

Related U.S. Application Data
(60) Provisional application No. 62/855,832, filed on May 31, 2019.

(51) Int. Cl. G01N 33/68; G16B 40/10

(52) U.S. Cl. CPC G01N 33/6848; G01N 2458/15; G16B 40/10

(57) ABSTRACT

Methods and systems for improved sample detection in mass spectroscopy are generally described. These are particularly useful, for example, for identifying a protein, a part of a protein, or a peptide when present in a low amount. In some embodiments, these can be useful to allow high-throughput proteomics studies for many samples, e.g., in series or in tandem. For example, certain embodiments are directed to novel approaches for identification of samples at the MS 1 level. In some cases, these improvements can be realized due to improvements in mass spectrometry instrumentation to better than the 1 ppm level for m/z measurements. Examples of improvements include, but are not limited to, improving internal mass standards, super-resolution peak fitting, isotopic labelling, Edman degradation and/or chromatography for proteins or peptides, and/or machine learning to predict peptide behavior, e.g., when exposed to such improvements.

### RELATED APPLICATIONS

This application claims the benefit of U.S. Provisional Patent Application Ser. No. 62/855,832, filed May 31, 2019, entitled “MS1-Based Peptide Identification for High-Sensitivity and High-Coverage Proteomics,” by Kirschner, et al., incorporated herein by reference in its entirety.

### TECHNICAL FIELD

Methods and systems for improved sample detection in mass spectroscopy, including for applications such as peptide processing and identification, are generally described.

### BACKGROUND

Mass spectrometry (MS) has become a leading protein analytical technique. Older techniques based on purely chemical methods for characterizing a single or small number of purified proteins can be effective in their capacity to identify and sequence proteins. However, though adequate for pure and abundant proteins, these methods can be laborious and not generalizable to mixtures of proteins or proteins of relatively low abundance. However, modern innovation through MS has been able to automate a general discovery tool for the rapid quantitative or semi-quantitative evaluation of thousands of proteins simultaneously, thus moving far beyond older techniques. There is now a demand for the quantitation of the individual proteins and an ability to identify and quantitate the presence and specific localization of myriad post-translational modifications.

Although RNA/DNA technologies have outpaced protein analysis in speed and cost, they have only increased the demand for very sensitive identification of proteins/peptides and their modifications. For example, there is increasing evidence that protein levels do not always correlate with mRNA, especially the dynamic regulation and modifications at the protein level that can be entirely missed in an RNA-based sequencing study. MS of a peptide sample involves correlating the mass of peptides with a look up table of protein sequences in an organism. In many cases, referencing the look up tables is performed automatically using computers. In theory, the “bottom up” matching algorithms ensure the identification of every protein through its multiple peptides. Limitations arise from the sheer complexity of the peptide sequences and the information provided by the single mass of the peptide. The yield of each peptide depends on the abundance of the protein in the mixture, the efficiency of cleavage, the efficiency of ionization. Furthermore, the identification of individual peptides is dependent upon the accuracy of the mass measurement and control of contaminating materials that give spurious mass peaks. In some cases, the peptides can carry a variety of different modifications which can further increase the complexity of the library of peptides to be identified.

While some current MS techniques may be adequate, much of biology has become focused on the study of regulatory proteins and on post-translational modification. There is a strong interest in understanding regulatory molecules, such as transcription factors, signaling proteins, membrane receptors, secreted factors, post-translational modification (PTM) enzymes such as kinases, sumoylation enzymes and other post-translational modifying enzymes and the reverse reactions mediated by phosphatases and other negative regulators, and current MS techniques are not adequate for studying these molecules due to their low abundance. In addition, identifying and quantifying these proteins as well as their various PTM remains an unsolved challenge. Accordingly, improvements in MS techniques are needed.

### SUMMARY

Methods and systems for peptide processing and identification are generally described. The subject matter of the present disclosure involves, in some cases, interrelated products, alternative solutions to a particular problem, and/or a plurality of different uses of one or more systems and/or articles.

In one aspect, the present disclosure is directed to a mass spectrometry method. In one set of embodiments, the method includes analyzing a sample using mass spectrometry to produce a sample data set; repeating the analyzing step one or more times to produce a plurality of plurality of sample data sets; and fitting corresponding peaks within the plurality of sample data sets to statistical distributions to determine the peak locations of the sample at super-resolution precision.

In another set of embodiments, the mass spectrometry method comprises dividing a sample comprising a peptide into at least a first portion and a second portion; isotopically labelling at least the first portion; analyzing the first portion using mass spectrometry; and analyzing the second portion using mass spectrometry.

The mass spectrometry method, in yet another set of embodiments, comprises dividing a sample comprising a peptide into at least a first portion and a second portion; applying Edman degradation to the peptide; analyzing the first portion using mass spectrometry; and analyzing the second portion using mass spectrometry.

In still another set of embodiments, the mass spectrometry method comprises applying a separation technique to a sample comprising a peptide to determine a separation parameter; analyzing the sample using mass spectrometry to produce a spectrum; and matching the spectrum and the separation parameter to a peptide dataset to determine the peptide.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A-1D are schematic representations of peptide identification process using MS1 and MS2 relative to using only MS1 in combination with certain techniques as described herein, in accordance with certain existing methods;

FIGS. 2A-2D are schematic diagrams in yet another embodiment of the disclosure;

FIGS. 3A-3C are schematic flow charts showing the division of a sample into a first portion and a second portion with subsequent labeling of one or both of the portions, according to some embodiments;

FIG. 4 is a table illustrating the results of using several methods described, comparing them to results obtained using MS1 and MS2, in another embodiment of the disclosure;

FIGS. 5A-5B are plots showing the use of super-resolution to identify the peptides within a bacterial lysate, according to some embodiments;

FIG. 6 is a plot of peptide identification incorporating amino acid counting combined with super-resolution mass analysis, according to some embodiments;

FIG. 7 is a plot comparing peptide identification with and without amino acid counting, according to one set of embodiments;

FIG. 8 shows a side-by-side comparison of peptide identification results with and without incorporating amino acid counting, in accordance with some embodiments;

FIG. 9 shows a side-by-side comparison of protein identification results with and without incorporating amino acid counting, in accordance with some embodiments; and

FIGS. 10A-10B are graphs illustrating peptide identification, in still other embodiments of the disclosure.

### DETAILED DESCRIPTION

Methods and systems for improved sample detection in mass spectroscopy are generally described. These are particularly useful, for example, for identifying a protein, a part of a protein, or a peptide when present in a low amount. In some embodiments, these can be useful to allow high-throughput proteomics studies for many samples, e.g., in series or in tandem. For example, certain embodiments are directed to novel approaches for identification of samples at the MS1 level. In some cases, these improvements can be realized due to improvements in mass spectrometry instrumentation to better than the 1 ppm level for m/z measurements. Examples of improvements include, but are not limited to, improving internal mass standards, super-resolution peak fitting, isotopic labelling, Edman degradation and/or chromatography for proteins or peptides, and/or machine learning to predict peptide behavior, e.g., when exposed to such improvements.

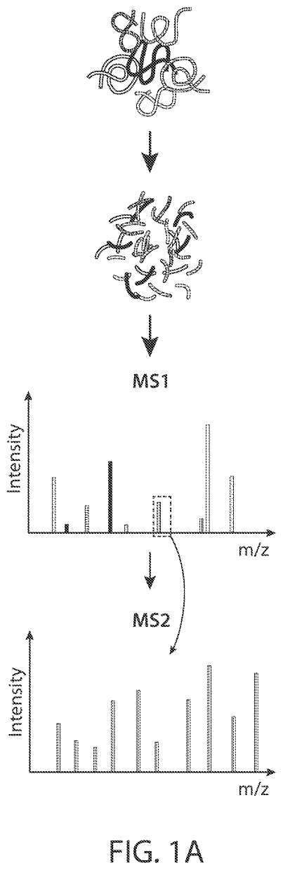

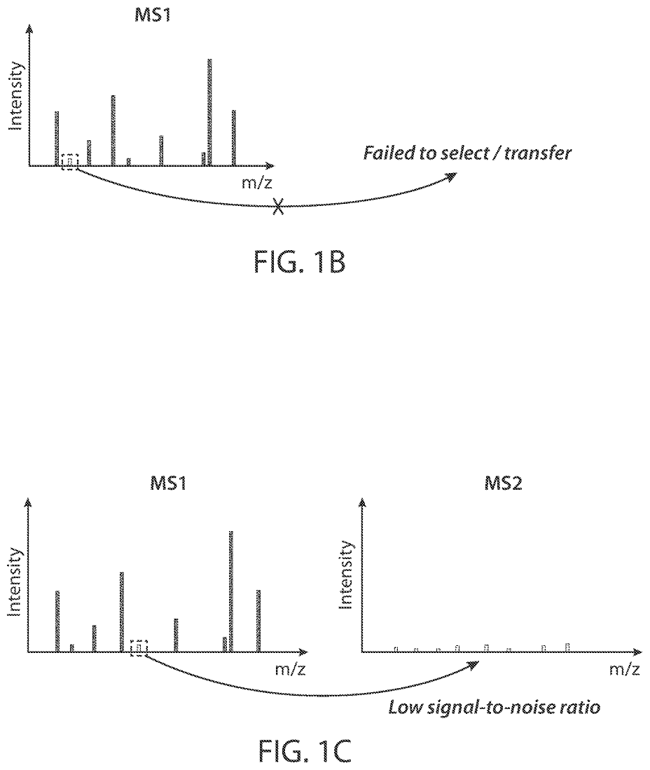

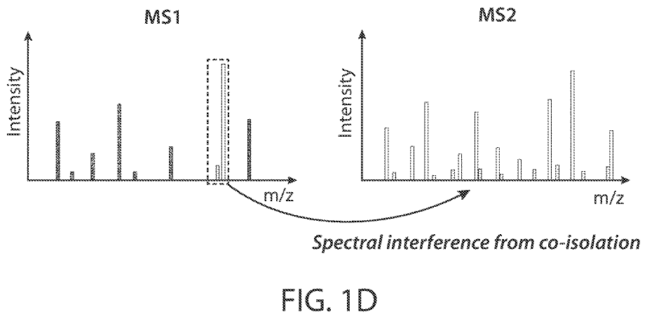

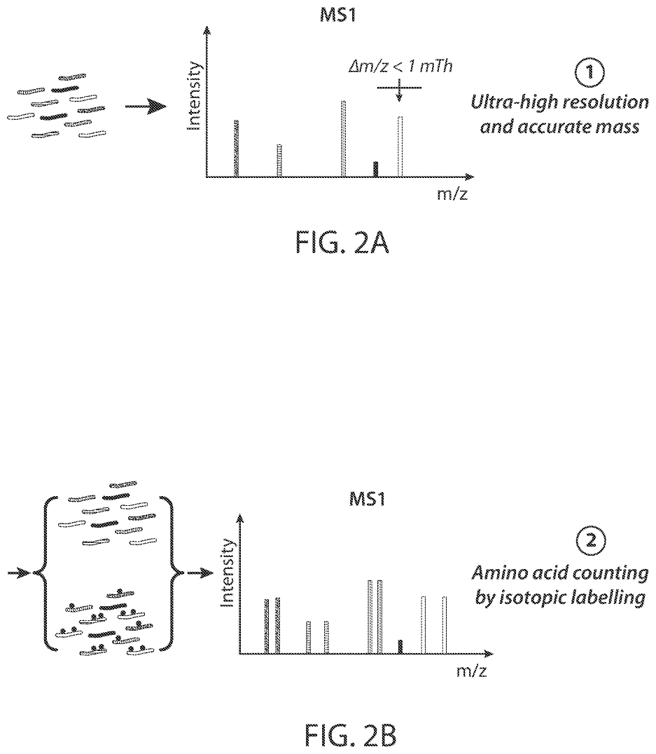

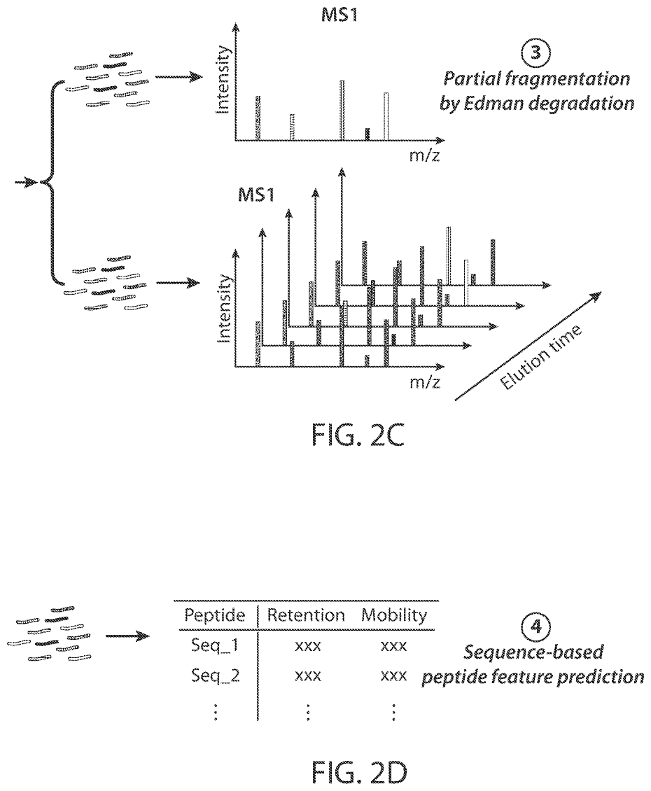

For example, various embodiments related to peptide identification and proteomic analysis are generally disclosed. In certain cases, systems and methods are described that use only a single mass spectrometer run or measurement (referred to by those of ordinary skill in the art as MS or MS1) as opposed to tandem mass spectrometry or MS/MS, where the stages are referred to as MS1 and MS2. For example, referring to FIG. 1A, a schematic illustration of a sample being analyzed by two mass spectrometers, MS1 and MS2, is provided, according to certain methods. In such systems, it may not be possible to apply an MS2 to an existing MS1 sample, as schematically illustrated in FIG. 1B, or the resulting MS2 data can have a low signal to noise ratio as schematically illustrated in FIG. 1C. In some cases, certain systems can carry spectral interference from co-isolated samples, as illustrated schematically in FIG. 1D. As such, some of the methods described herein can improve upon these shortcomings. For example, in reference to FIG. 2A, super-resolution (e.g., ultra-high resolution) mass data of a sample can be obtained from just a single MS1. In some embodiments, a sample can be compared to an identical sample that has been labeled, as schematically illustrated in FIG. 2B.

Some methods disclosed herein may improve the quality of peptide identification data from just one mass spectrometer run. However, it should be noted that while some of the methods described herein may be used with data from a single mass spectrometer run, in some cases, more than one mass spectrometer run may be used (e.g., as in tandem mass spectrometry, or other techniques), such that the quality of data (e.g., resolution or mass accuracy of a peptide) obtained is improved as the sample is processed by two or more mass spectrometer runs, i.e., the systems and methods described herein are not limited to only use with MS1 techniques.

The methods described herein may, in some aspects, provide quantitative data (mass, mass-to-charge ratio, etc.) about various samples, including the identity of a peptide or peptides that make up a protein. Other types of samples are discussed in more detail below. In certain cases, the methods described herein may advantageously identify peptides, or other samples, even when only a low concentration and/or a low amount of sample is provided. In some embodiments, the amount of sample is less than 100 picograms, or other amounts as discussed herein. Accurately determining relatively low (e.g., 100 picograms or less) has persisted as a challenge in the field of proteomics and mass spectrometry. Advantageously, mass spectrometry methods described herein can be used in some cases to determine the mass of peptides in a sample as small as 100 picograms. Some embodiments are especially advantageous when identifying relatively small or subtle changes in a sample. For example, post-translation modifications of a peptide may be rare and/or may not result in large changes in mass or mass-to-charge ratio, etc., such as for certain regulatory peptides. In this way, accurate, precise, and/or quantitative data can be obtained from one mass spectrometer measurement (e.g., MS1), achieving much higher degrees of detection with only a low amount of sample, in accordance with some embodiments.

In some embodiments, a mass spectrometer is used to analyze a sample. A mass spectrometer (MS) is an instrument used in mass spectrometry, the latter being an analytical technique that, as known in the art, measures the mass-to-charge ratio (m/z) of ions and can be used to determine the chemical identity of atoms, molecules, peptides, proteins, and other samples, such as those described herein. As mentioned, MS1 can refer to a mass spectrometry technique using a single mass spectrometer run or measurement, e.g., in contrast to tandem mass spectrometers and the like (which stages are often referred to as MS1 and MS2). In some embodiments, the systems and methods described herein can be applied to a single mass spectrometer analysis (MS1), e.g., to improve identification of samples at the MS1 level, although more than a single mass spectrometer run may be used in other embodiments.

A mass spectrometer typically uses an ionization technique in order to vaporize a sample. In certain embodiments, electrospray ionization (ESI) is used ionize the sample. ESI is used to produce ions in an electrospray to which a high voltage is applied to a liquid sample (e.g., a solution) to create an aerosol, as is known by those of ordinary skill in the art. Certain mass spectrometry embodiments may use other methods of ionization, such as atmospheric pressure chemical ionization (APCI) or matrix-assisted laser desorption ionization (MALDI). Still other ionization methods are possible and those of ordinary skill in the art in view of the teachings of this disclosure will be able to select an appropriate ionization method to maximize or minimize peptide fragmentation for the desired peptide identification.

Certain embodiments ionize a sample (e.g. peptide, protein, etc.) into the gas phase and determine the charge-to-mass ratio (m/z) of an ion by analyzing the species' behavior in a mass analyzer. A mass analyzer is an instrument (or part of an instrument) that uses the behavior of an ion in the gas phase to determine the mass-to-charge ratio of the species. In some embodiments, the mass detector is a quadruple mass detector. The quadrupole mass detector, in some embodiments, uses four parallel metal rods where each opposing rod pair is connected together electrically, and a radio frequency voltage with a DC offset voltage is applied between one pair of rods and the other. Ions can travel down the quadrupole between the rods, and ions of a certain mass-to-charge ratio reach the detector for a given ratio of voltages, while other ions have unstable trajectories and will collide with the rods. This permits selection of an ion or ions with a particular m/z or allows for the scanning of a range of m/z-values by continuously varying the applied voltage. Other mass analyzers may be suitable, such as a time-of-flight (TOF) analyzer may be used.

Certain embodiments as described herein are based on improvements in techniques for determining the charge-to-mass ratio. For example, in some embodiments, improvements of better than 100 ppm, better than 50 ppm, better than 30 ppm, better than 10 ppm, better than 5 ppm, better than 3 ppm, better than 1 ppm, or better than 0.5 ppm for m/z measurements can now be achieved. It should be understood that “ppm” is used in reference to relative amounts, e.g., for a peptide with 1000 Da, 1 ppm would be 0.001 Da. In some cases, mass spectrometers exhibiting such improved m/z measurements can be obtained commercially. Such improvements can be used, for example, in conjunction with techniques such as improved internal mass standards, super-resolution peak fitting, isotopic labelling, and/or other analytical techniques such as Edman degradation, chromatography, etc., e.g., as discussed herein, to improve analysis of samples, for example, at the MS1 level.

Such improvements can be used, for example, to detect relatively low amounts of sample. In some embodiments, the amount of sample may be equal to or less than 100 nanograms, less than 50 nanograms, less than 30 nanograms, less than 10 nanograms, less than 5 nanograms, less than 3 nanograms, less than 1000 picograms, less than 500 picograms, less than 300 picograms, less than 100 picograms, less than 50 picograms, less than 30 picograms, etc. As discussed below, due to such improvements, more “peaks” may be determined with mass spectrometry, e.g., without missing peaks caused by insufficient amounts of sample, smaller MS peaks, or the like. In addition, such improvements may allow for better resolution of peaks that are closely packed together. This can be further improved, for example, using techniques such as super-resolution peak fitting, or the like, e.g., as discussed herein.

A variety of samples can be determined. For example, in certain embodiments, the sample to be analyzed is a biological sample. Non-limiting examples of biological samples include proteins, enzymes, peptides, regulatory molecules, nucleic acids (e.g., DNA, RNA), lipids, polysaccharides, metabolites, and carbohydrates. Other biologically relevant molecules are also possible. For certain embodiments, the biological sample is a single cell. Since some of the embodiments as described herein may be advantageously beneficial in identifying even small amounts of peptide, such as noted above, detecting peptides associated with one cell may be achieved. For certain applications, detecting the presence of very low amount of certain peptides, such as biomarkers, or MHC presented cancer antigens, may be achieved.

As a specific example, in some cases, systems and methods described herein may be advantageously useful for identification of molecules attached to a peptide after translation (e.g., post-translational molecules). These may be understood to be molecules that are bound to a peptide or protein after the process of translation, sometimes known as a post-translational modification (PTM). In some cases, post-translational molecules that can be analyzed, e.g., as described herein, are rare and are only present in low amounts or concentrations. As noted above, however, a variety of different modification, e.g., to proteins, peptides, and other molecules, may be determined, qualitatively or quantitatively, such as is discussed herein.

For example, in certain aspects, a sample may be modified prior to being processed. For instance, the sample, or a portion thereof, may be modified in a way as to change its atomic weight. As a non-limiting example, in some embodiments, a sample is modified with an isotope of an atom already present within the sample (i.e., isotopic labeling). In some embodiments, the sample modified with an isotope may be compared with an identical sample unmodified with an isotope so that information about the peptide may be gained. Non-limiting examples of isotopes include ²H (D or deuterium), ¹³C, ¹⁵N, etc. In some cases, labeling compounds may be used that include such isotopes (e.g. heavy amino acids, NeuCode amino acids, D-modified maleimide, heavy variants of TMT and other NHS-based labeling moieties, etc.).

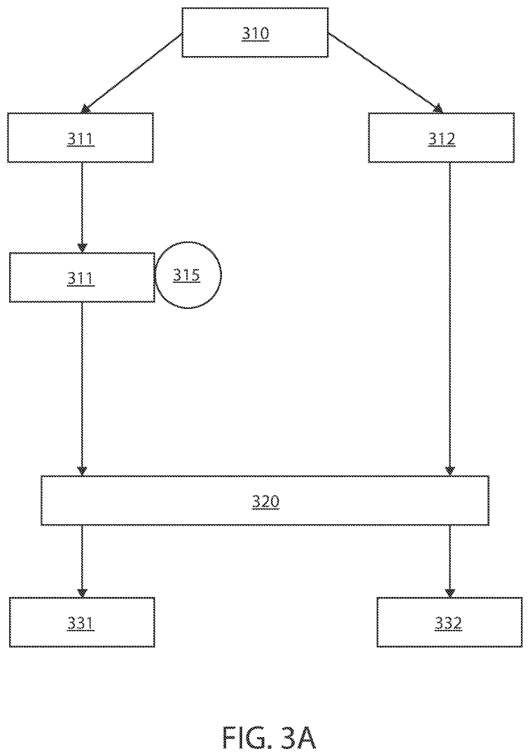

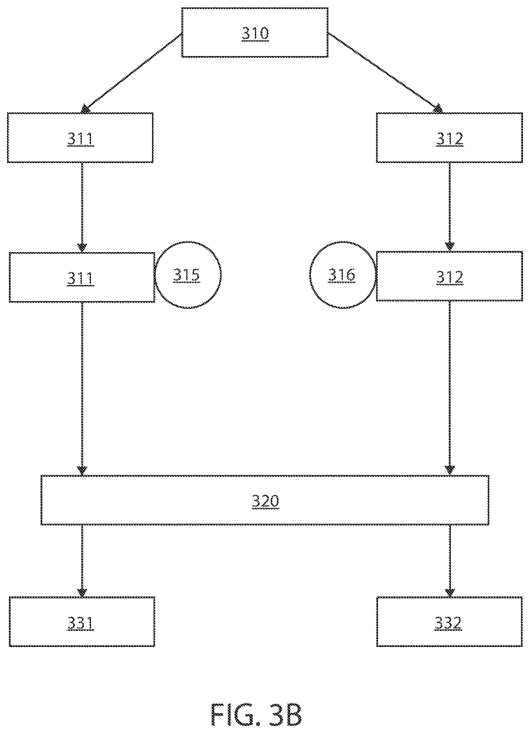

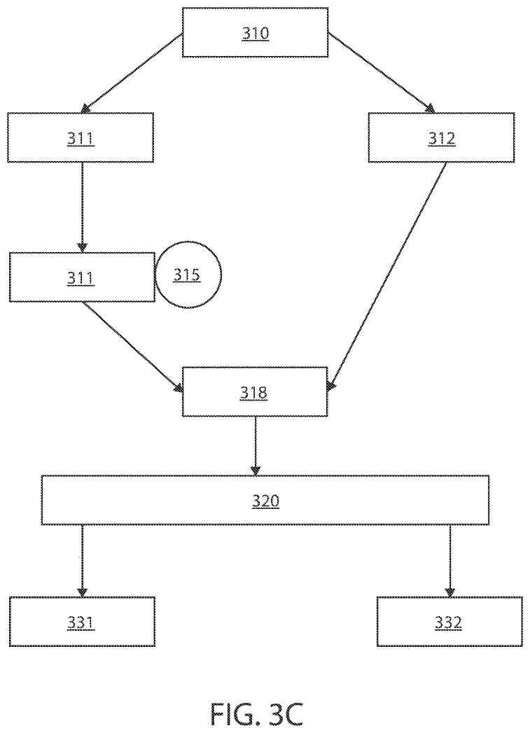

Thus, in some embodiments, a sample can be divided into two (or more) portions, and the samples differently modified or labelled. For example, the samples may be modified to have different masses, or using techniques such as those described below. In reference to FIG. 3A, in accordance with some but not all embodiments, a sample 310 can be divided into a first portion 311 and a second portion 312. Either first portion or the second portion can be labeled in order to change the mass of the sample. For example, in FIG. 3A, first portion 311 has been labeled with label 315. The sample may then be analyzed using MS. The first and second portion can then be subjected to a single mass spectrometer, such as mass spectrometer 320. The resulting mass spectra, mass spectrum 331 for first portion 311 and mass spectrum 332 for second portion 312 can then be compared in order determine mass information about the components (e.g., peptides) of sample 310. In some embodiments, both the first portion and the second portion can be labeled. For example, in FIG. 3B, first portion 311 is labeled with label 315 and second portion 312 is labeled with label 316. In some embodiments, labels for a particular portion (e.g., a first portion, a second portion) are different. In some cases, the samples may be recombined prior to MS analysis. For example, in reference to FIG. 3C, labeled first portion 311 and second portion 312 can be recombined into a recombined sample 318. Two samples may produce a pair of peaks, whose mass difference is reflective of the differences in labeling, which can be used to determine the sample. This can be extended to multiple samples as well (e.g., 3 modifications or labels to produce a triplet of peaks). It should also be understood that this principle can be applied more than once (for example, to different amino acids within a peptide), e.g., simultaneously, sequentially, combinatorically (e.g., splitting into more than two samples and their associated peaks in MS), etc. The same or different techniques can be used each time.

For example, as described above, in one set of embodiments, a sample (or portion thereof) may be modified by adding or modifying the sample, e.g., with a label. Examples of labels include different isotopes, different chemical modifications, different side groups, or the like. Examples include nucleic acids, peptides, or polysaccharides, etc. As another example, an internal mass standard may be used. The standard may, in some cases, be one that is stable over time, and one which gives a high signal-to-noise ratio, which may allowing for accurate mass measurement and calibration. In some embodiments, the internal mass standard is a compound that is externally introduced to sample (e.g., protein, peptide) prior to an MS1 run and has a known, fixed mass. In some embodiments, the internal mass standard comprises ions originating from the same peptide or protein of the sample, but with a different charge. In some cases the internal standard may have a controlled m/z ratio. In some cases, one or more internal mass standards could facilitate an increase in mass measurement resolution, accuracy, and/or provide better calibration and/or normalization across an entire spectrum, and/or across a wide m/z range.

In addition, for peptides, the peptides may be modified in some fashion prior to MS analysis. For example, a peptide may be at least partially degraded, e.g., using techniques such as Edman or Bergmann degradation. Such techniques may, for example, produce samples having different masses (corresponding to differences in amino acid sequence due to degradation), which can be determined using MS, e.g., using MS1.

For instance, in some cases, a sample can have a peptide modified by Edman degradation. Edman degradation is known in the art as a method of sequencing amino acids in a peptide by reacting the N-terminal amino group with phenyl isothiocyanate under mildly alkaline conditions to form a cyclical phenylthiocarbamoyl derivative. Then, under acidic conditions, this derivative of the terminal amino acid is cleaved as a thiazolinone derivative. The thiazolinone amino acid is then selectively extracted into an organic solvent and treated with acid to form the more stable phenylthiohydantoin (PTH)-amino acid derivative that can be identified by using chromatography or electrophoresis. This can then be repeated again to identify the next amino acid. Information gained from this process, in some embodiments, can help identify a peptide in combination with methods described herein. In certain embodiments, Edman degradation is applied to at least a first portion of a sample comprising a peptide in order to compare to an identical sample that is absent in Edman degradation in order to gain information about the identity of a peptide. Other non-limiting examples of peptide modification include enzymatic and chemical approaches. Examples of chemical approaches include, but are not limited to, BrCN cleavage. Examples of enzymatic approaches include, but are not limited to, digestive enzymes, such as trypsin, chymotrypsin, lysC, gluC, etc.

In some embodiments, a sample can be processed or run multiple times in the mass spectrometry with different parameters. As non-limiting examples, in some cases, the sample can be run under different ionization voltages, either in an alternating form (e.g. high, low, high, low, . . . ) in consecutive MS1 scans, or in separate MS1 runs in tandem, and/or with more number of parameters, and/or longer defined sequences of parameter settings (e.g., v1, v2, v3, v4, v1, v2, v3, v4, . . . ), to help extract information regarding the sample. This may be combined with other information, e.g., as discussed herein, to further reduce sample complexity and/or improve confidence in identification of the sample.

In one set of embodiments, a sample may be analyzed or modified using other techniques, e.g., prior to MS analysis. For example, in some cases, information about the identity of proteins or peptides to be identified may be obtained along with MS analysis. Such information can be obtained before, during, or after MS analysis.

In some embodiments, a separation technique is applied to a sample comprising a peptide to determine a separation parameter. As an example, in certain embodiments, information may be provided by a liquid chromatography (LC) system as the separation technique associated with the mass spectrometer. Accordingly, in some embodiments, the separation parameter comprises elution time. For example, in reference to FIGS. 2C and 2D, a sample can be run using MS1, and the same sample can also be run through an LC in order to obtain the elution time or the retention time of the sample. The information gained from running a sample through a chromatography column and extracting the retention time and/or the elution, in some cases can be computationally predicted from at least one parameter (e.g. peptide sequence, amino acid composition, charge, pI, size, polarity, etc., as non-limiting examples) can be determined, and in some cases, can be combined with information obtained from the MS analysis in order to help identify a sample. In certain embodiments, high-performance liquid chromatography (HPLC) or another method of chromatography is used as the separation technique. In this way, samples such as proteins or peptides may be at least partially separated prior to entering a MS instrument. In some cases, the separation method associated with the MS may also introduce the sample into the MS to facilitate processing or analysis of the sample, for example, an LC system connected to a mass spectrometer.

As another example, in certain embodiments, information may be provided by a field asymmetric ion mobility spectrometry (FAIMS) device associated with the mass spectrometer. The information gained from running a sample through a FAIMS device and a prediction, for example, voltage, which may be computationally predicted from at least one parameter (e.g. peptide sequence, amino acid composition, charge, pI, size, polarity, etc., as non-limiting examples) can be determined, and in some cases, combined with information obtained from the MS analysis in order to help identify a sample. In this way, samples such as proteins or peptides may be at least partially separated prior to entering a MS instrument, according to some embodiments. In some cases, the separation method associated with the MS may also introduce the sample into the MS to facilitate processing or analysis of the sample, for example, an FAIMS system connected to a mass spectrometer.

Identification of a sample, such as a peptide or a protein, may be accomplished, in full or in part, in some embodiments, using algorithms or software to analyze the mass spectroscopy data. For example, in some cases, fragmentation or peak pattern(s) can be obtained from MS1, and analyzed at charge-to-mass ratios such as those discussed herein. In some cases, differences that result in peak splitting or other changes (e.g., caused by internal mass standards, isotopic labelling, sequencing or degradation, chromatography, etc.) may be determined to determine the sample. For instance, such measured patterns may be compared to established patterns, e.g., in a dataset, to determine matches between measured and established patterns, which can be used to identify which molecules (or portions thereof) are present within the sample. The established patterns may be determined, for example, experimentally, and/or via computer modeling. The matches may also be full or partial, depending on the application. In some cases, techniques such as machine learning, artificial intelligence, or other computer matching algorithms may be used to determine matches (which may include partial matches). In some embodiments, such techniques may use or combine data from different inputs, e.g., other analytical techniques such as those discussed herein. These may include chemical information obtained by HPLC, fragmentation data obtained by MS1, a database with known protein or peptide identification parameters, or other sources of data.

In some cases, super-resolution techniques may be used to analyze the mass spectroscopy data. In some cases, this may result in higher m/z resolutions and accuracies than the values reported by the MS instrument itself or current standard analysis methods. For example, in some embodiments, a plurality of mass spectroscopy analyses of a sample may be obtained, e.g., resulting in a plurality of sample data sets (e.g., intensity vs. m/z), and peaks from the plurality of sample data sets may be fitted to statistical distributions to determine the peak m/z precisions, and in some embodiments, the relationship between each individual peak's intensity and m/z resolution. For example, the statistical distributions of peaks arising from adjacent or other MS1 scans may be fitted (e.g., curve fitting) to Gaussian, elliptical Gaussian, or other distributions (for example, an x exp(−x) distribution), and the maxima of the distribution may be used as the expected or idealized estimates of resolutions of the peaks in consideration. For instance, curve fitting can be used to extract mass peaks at a resolution that is finer than what is provided (e.g., recorded) by MS1 instrument alone. Curve fitting (e.g., Gaussian fitting) can be performed on neighboring mass values of a particular peak in the mass spectrum, as well as combining temporally adjacent mass measurements. Advantageously, curve fitting as described herein can be combined in some embodiments with internally-calibrated and/or peak-dependent precision measurements, and in some cases, additional mass calibrations can be performed in addition to the mass calibration standards within the instrument in order to provide an increase in the mass precision. In some embodiments, m/z determination and resolution measurement could be differently performed for each individual peak, giving higher confidence to peaks with higher m/z resolution. In some cases, this may result in the identification of peaks at resolutions that are higher resolutions than the resolution imposed by the MS instrument itself. In some cases, at least 3, at least 5, at least 10, at least 30, at least 50, or at least 100 measurements of a sample may be used to produce the plurality of sample data sets for super-resolution analysis.

In some embodiments, a super-resolution technique can comprise obtaining mass values from at least one MS1 scan and then obtaining subsequent scans (i.e., neighboring scans) of the same or different sample as well as from different isotopic peaks that can then be grouped together and their pairwise differences can be calculated. Advantageously, the individual MS1 scans can be of a high-resolution or low-resolution. In some cases, by combining the MS1 mass values with data from neighboring scans, the accuracy of the mass values can be improved to provide super-resolution mass data, often with just a single mass spectrometer run (e.g., MS1). These mass values can then be used to model the measurement precision based on an expected error distribution (e.g., a Gaussian, or other distributions such as those described herein), which can return a peak-dependent precision value.

In some embodiments, intensity-based mapping can be used. This can be particularly advantageous, for example, in cases where a peak intensity is weak (e.g., having too few consecutive frames, too few isotopes measured reliably), This mapping can be generated by pooling the statistics of all the peak-dependent precision values determined by the entire dataset (e.g., a scan with its neighboring scans), which can establish a square root dependence between the measured peak intensity and precision value. The result of such intensity mapping can be peak-dependent in some cases, and/or can provide a more reliable and complete mass measure than methods that use a fixed value, bootstrapping method, or any formula-based estimate.

Super-resolution techniques as described herein can also be combined with the use of labeling techniques described herein in accordance with certain embodiments, as well as used in some cases with internal mass calibration standards such as those described herein to improve the mass determination of a sample. In some embodiments, long-range mass calibration can further be enhanced by combining the peak-based mass calibration and super-resolution techniques described herein.

U.S. Provisional Patent Application Ser. No. 62/855,832, filed May 31, 2019, entitled “MS1-Based Peptide Identification for High-Sensitivity and High-Coverage Proteomics,” by Kirschner, et al., is incorporated herein by reference in its entirety

The following examples are intended to illustrate certain embodiments of the present disclosure, but do not exemplify the full scope of the disclosure.

### Example 1

This example describes in silico peptide measurement and identification in accordance with one embodiment of the disclosure.

In silico peptide measurements and identification analyses of proteins were performed using the UniProt Human Proteome Databasae (UP000005640_9606) supplements with isoforms, and performed in silico trypsin digest with an allowed maximum skip of 2. Peptide variants with methionine oxidation, phosphorylation on serine and threonine, N-terminal acetylation, and lysine trimethylation were included, all treated as dynamic modifications to represent common post-translation modifications on proteins. All ions generated from these peptides were calculated and compiled with charges up to z=6 in searching the database.

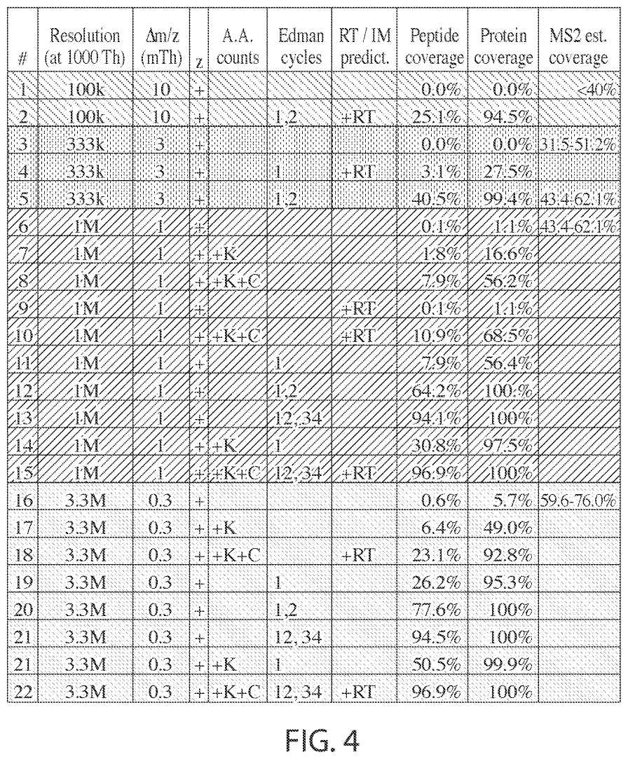

Approximately 3000 randomly chosen peptides from across the database were selected. FIG. 4 shows the percentage of unique peptide and protein identification for various implementations and combinations of this example. As shown in FIG. 4, higher mass accuracy significantly reduces the library complexity and identification degeneracy. However, with mass and charge identification alone, only a very low fraction of peptides was identified (from 0.0% at m/z tolerance of 3 mTh (millithomsons) to almost 0.6% at 0.3 mTh, see rows 3, 6 and 16). With the inclusion of each of the extra peptide-level information (e.g. amino acid counting for lysine and cysteine (K/C), Edman degradation, and retention time and/or ion mobility prediction), the fraction of uniquely identifiable peptides is significantly improved (see rows 7-9, 11-13). Specifically, for m/z tolerance at 1 mTh (equivalent to 1 M resolution at 1000 Th), with the combined use of K/C counting and retention time prediction, 10.9% of peptides can be uniquely identified at the MS1 level (see row 10); with two rounds of Edman degradation (and three separate MS1 sessions), 64.2% unique identification for single-cycle and 94.1% for dual-cycle runs (see rows 12 and 13) were achieved. Various combinations of these methods (see rows 10, 14 and 15), more amino acid counting, more Edman degradation cycles, and even higher mass accuracy can all further increase the identification percentage and robustness.

It is noted that these coverages are calculated at the peptide level, which translate to much higher coverage at the protein level, assuming that multiple peptides are efficiently ionized and detected. For example, assuming 10 peptides detected for each protein, a 7.9% unique identification coverage at the peptide level (with K/C counting at 1 mTh tolerance, see row 8) translates to a high 56.8% identification rate at the protein level; and 30.8% peptide identification (with K counting and one cycle of Edman degradation at 1 mTh, see row 14) translates to a very high 97.5% protein identification.

For a direct comparison with the performance against MS2-based (or MS/MS-based) peptide assignment, the percentage of MS2 unique coverage at the same levels of mass accuracy and filtering were estimated with the assumption that 50% fragment peak ionization and detection efficiency, and 20-30% distinct fragment peaks were required for robust identification at the MS2 level (i.e. 4-8 distinct peaks needed from the correct peptide per each 10 a.a. (amino acid) length; in practice the median number of distinct peaks from rank 1 peptide against rank 2 is roughly 8 per 10 a.a.). With these assumptions, MS2 estimations and identification were taken under a similar mass accuracy to range from 5.3-51.2% (at 3 mTh) for to 43.4-62.1% (at 1 Th). It is noted that, various combinations in this example achieved similar levels of peptide identification with MS1 level information only (e.g. see rows 5, 12 and 14), and certain combinations of showed much higher identification rate (e.g. see rows 13 and 15).

### Example 2

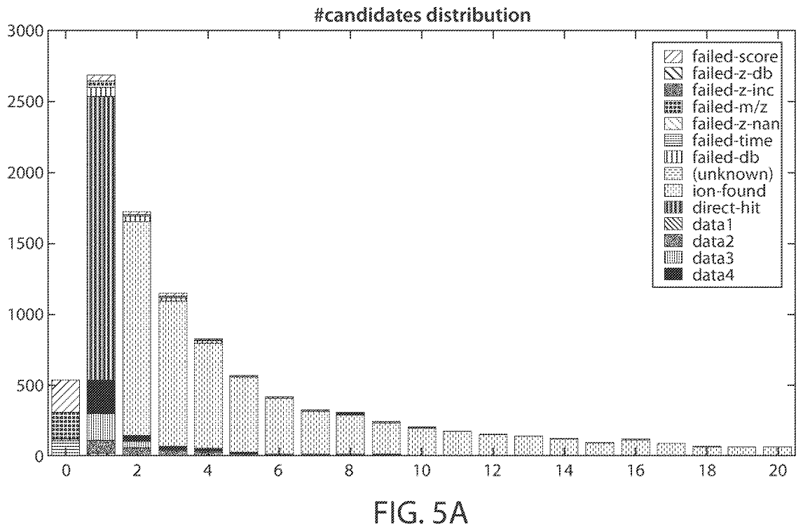

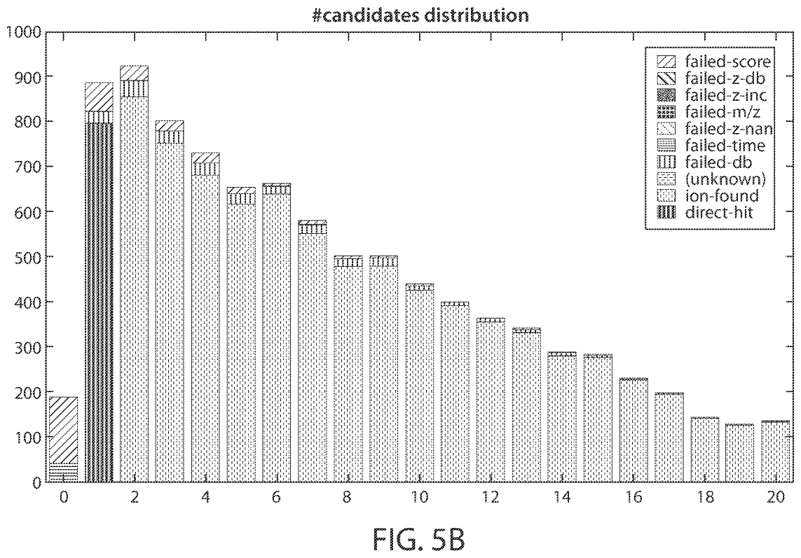

This example shows identification results for a 500k resolution human cell lysate MS/MS data, removing the set of peptides successfully identified by MS2. This results in information from only MS1. These data sets assume that the number of lysines and cysteines can be determined. The retention time is then predicted. FIG. 5A uses 5 ppm mass error, while FIG. 5B uses 1.5 ppm mass error.

FIG. 5A illustrates that 7.2% of compounds, including 89.3% of the peptides, were correctly identified. FIG. 5B illustrates that 22.9% of compound, including 94.5% of the peptides, were correctly identified.

FIGS. 5A-5B show this MS1-based analysis results for human cell samples. The histograms show, out of a few thousands MS2-identified peptides, what are the chances and correctness that they can be identified with one particular embodiment of the disclosure. The x axis is degeneracy (i.e., for each peptide in question, with MS1 information, a peptide can be narrowed down to x choices), and y is peptide count (i.e., how many peptides can be identified with x choices). Thus, the height of the second bar (x=1) indicates the total number of peptides that can be narrowed down to a single choice, out of which, the highlighted ones indicates those that are identified correctly. The percentages thus illustrate how many peptides can be identified (e.g., 7-30%) and how many of those are correct (90-95%) in these experiments. These results establish confidence that complex human proteome samples can be analyzed. Note that these numbers are per peptide, and the per protein values will be much higher.

These samples were essentially prepared as described in Wühr, et al., Current Biology: 2015, 25, 2663-2671, incorporated herein by reference. BL21 DE3 Escherichia coli cells were grown to an OD of 1.0. Cells were pelleted and lysed in 6 M guanidine hydrochloride, 50 mM HEPES pH=7.4. Disulfide bonds of ˜500 micrograms of protein were reduced with 5 mM DTT (500 mM stock, water) at 60° C. for 20 min. Samples were cooled to room temperature and cysteines were alkylated by the addition of 15 mM N-ethyl maleimide (NEM) (1 M stock, acetonitrile) at 23° C. for 20 min. 5 mM DTT (500 mM stock, water) was added at 23° C. for 10 min to quench any remaining NEM. Salts, small molecules and lipids were removed by a methanol-chloroform precipitation and the protein disc was washed with 50/50 methanol/chloroform one additional time and the protein was allowed to air dry. Protein samples were dissolved in 6 M guanidine hydrochloride, 10 mM EPPS pH=8.5 to ˜2.5 micrograms/microliter. Samples were heated at 60° C. for up to 30 minutes to help resolubilization. Next, samples were diluted with 10 mM EPPS pH=8.5 to 2 M guanidine hydrochloride. Lysates were digested overnight at 37° C. with LysC (Wako, 2 micrograms/microliter stock in HPLC water) at a concentration of 10 ng/microliter LysC. Samples were further diluted to 0.5 M guanidine hydrochloride with 10 mM EPPS pH=8.5 and an additional 10 ng/microliters LysC was added as well as 20 ng/microliters of sequencing grade Trypsin (Promega). Samples were mixed by pipetting and incubated at 37° C. for 12-16 hours. All solvent was removed in vacuo and samples were re-suspended in HPLC water at 0.2 micrograms/microliter of peptides. 20 micrograms of peptides were acidified to pH <2 with HPLC triflouroacetic acid and a stage-tip was performed to desalt the samples.

### Example 3

The following example describes the analysis of peptide sample using super-resolution fitting, amino acid counting, and combinations of the two.

A peptide digestion and identification based on an MS1 method, as described by this disclosure, are used using a sample from a bacteria lysate. Bacteria peptide sample was prepared using SILAC labelling with K0/K+8 and RO/R+10 isotopic labels, cysteine was protected by iodoacetamide. The sample was run on a Thermo Orbitrap Lumos Tribrid mass spectrometer, with a 120 min LC gradient, 500k mass resolution. The set of unique MS/MS identified peptides was used as the ground truth dataset (as produced by MaxQuant). However in the identification procedure, no information from the MS/MS scans was used. The identification used the following parameters: ion charge range: 1-8, max allowed missing cleavages: 2, differential modifications considered: methionine oxidation, N-terminus acetylation, N-terminal methionine removal. A custom soft-clipping scoring function algorithm was used, and identification was reported only when highest candidate score is higher than the second one by a fixed threshold.

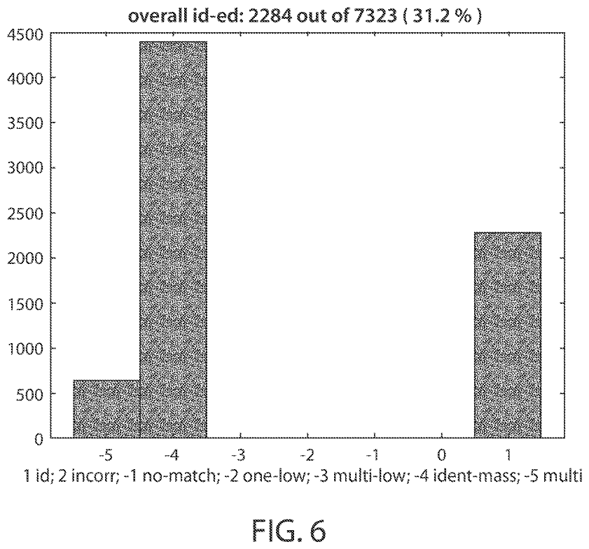

FIG. 6 shows peptide identification using accurate super-resolved mass peaks only. Different identification results are summarized in seven categories along the (X axis) of FIG. 6: (1) identifications which are of the correct mass (2) identifications which is incorrect (in this case the count is 0, therefore not shown), (−1) no matching database entry found, (−2) one candidate found, which did not pass the threshold, (−3) multiple candidates found, and the highest one didn't pass the threshold (−4) more than one candidates with identical mass found, and (−5) multiple candidates found with non-identical mass. The analysis technique uniquely identified 31% of all peptides in this database.

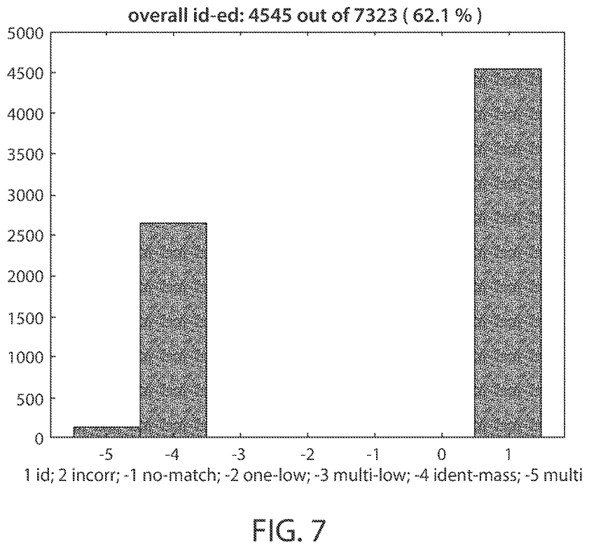

FIG. 7 shows peptide identification by incorporating amino acid counting (lysine and arginine, or KR counting) on top of accurate super-resolved mass. Different identification results are summarized in seven categories (X axis), as above. The uniquely identified 62% of all peptides in this database, doubling the ratio from the case above.

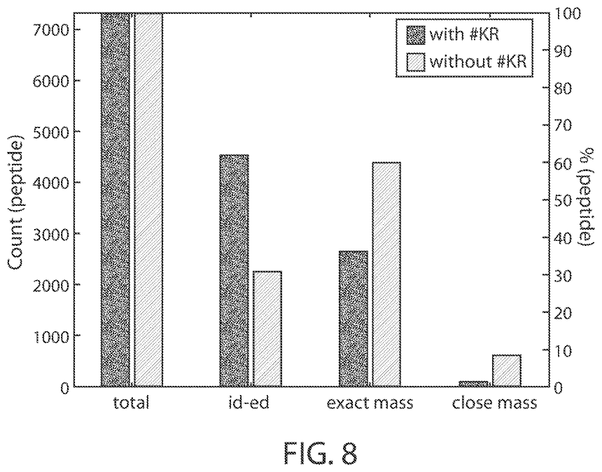

FIG. 8 shows a side-by-side comparison of peptide identification results with and without incorporating amino acid counting (lysine and arginine, or KR counting), on top of accurate super-resolved masses. Different identification results are summarized in three categories (X axis), “id-ed”: unique identification, “exact mass”: lack of identification due to presence of more than one peptide with identical mass, and “close mass”: lack of identification due to presence of other peptides with similar but non-identical mass. The incorporation of KR counting data significantly decreased the fraction of “exact mass” peptides, thus allowing much higher rate (doubled) of unique identification.

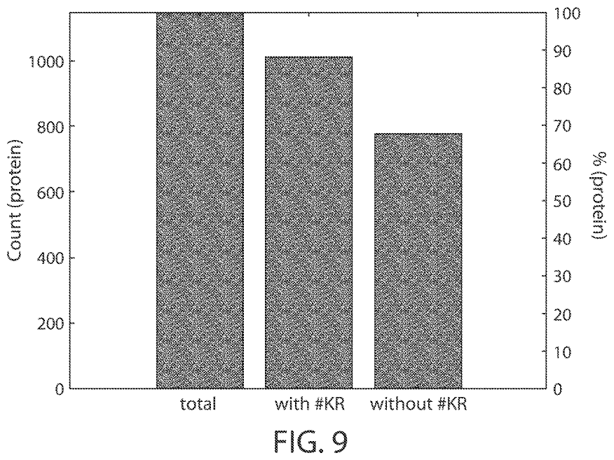

FIG. 9 shows a side-by-side comparison of protein identification results with and without incorporating amino acid counting (lysine and arginine, or KR counting), on top of accurate super-resolved mass. Each protein is considered identified if at least one of its peptide digestion products is identified. As a result, our method has identified a much higher percentage of proteins (than percentage of peptides), covering 90% of all identified proteins by MS/MS method) with KR counting.

### Example 4

The following example describes a peptide digestion and identification based on the MS1 methods described elsewhere herein using a bacteria lysate sample.

The bacteria peptide sample was prepared using SILAC labelling with K0/K+8 and RO/R+10 isotopic labels, cysteine was protected by iodoacetamide. The sample was run on a Thermo Orbitrap Lumos Tribrid mass spectrometer, with a 120 min LC gradient, 500k mass resolution.

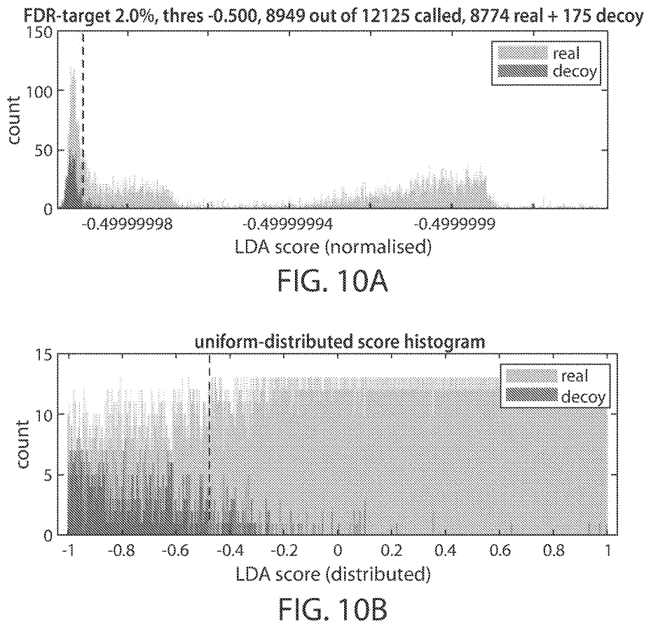

A set of all MS/MS identified peptides was used as a comparison dataset (as produced by MaxQuant). However, in this procedure, no information is used from the MS/MS scans. The mass identification used the following parameters: ion charge range: 1-8, max allowed missing cleavages: 2, differential modifications considered: methionine oxidation, N-terminus acetylation, N-terminal methionine removal. Accurate super-resolved mass, KR counting information, as well as retention time predictions were used for the analysis. The iRT retention time prediction algorithm was also used with an additional custom re-normalization step. A custom soft-clipping function for candidate scoring were also used. A custom decoy database that preserves the library size as well as peptide mass and length distribution by swapping the last amino acid in each peptide with the first in the preceding peptide was also utilized in this example. A quadratic discriminant analysis was used to build the scoring model shown in FIGS. 10A-10B, incorporating features including peptide length, missed cleavages, charge, intensity, m/z, Δ(m/z), RT, Δ(RT), RT_fwhm, score, and Δ(score).

FIGS. 10A-10B show the distribution of peptide scores from the discriminant analysis model. Top, normalized scores, bottom, distributed scores. Peptides from real and decoy databases are shown in two different shadings. By regulating the false discovery rate (FDR) to 2, our method identified 63% of MS/MS identified peptides (10412 out of 16458), of which 8949 out of 12125 were unique, accounting for 74% of all peptides. By incorporating a matching decoy peptide library to the correct target library, and determining what is the expected rate of erroneous peptide assignment, an FDR (false discovery rate) framework can be provided. With the method and a custom FDR algorithm, at an FDR level of 2%, 63% of MS/MS identified peptides (10412 out of 16458) were identified, of which 8949 out of 12125 were unique, or 74% of peptides.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A mass spectrometry method, comprising:
analyzing a sample using mass spectrometry to produce a sample data set;
repeating the analyzing step one or more times to produce a plurality of sample data sets; and
fitting corresponding peaks within the plurality of sample data sets to statistical distributions to determine the peak locations of the sample at super-resolution precision.

2. The method of claim 1, further comprising internally calibrating the corresponding peaks.

3. The method of claim 2, further comprising calibrating mass standards of the sample data set using the corresponding peaks.

4. A mass spectrometry method, comprising:
dividing a sample comprising a peptide into at least a first portion and a second portion;
isotopically labelling at least the first portion;
analyzing the first portion using mass spectrometry; and
analyzing the second portion using mass spectrometry.

5. A mass spectrometry method, comprising:
dividing a sample comprising a peptide into at least a first portion and a second portion;
applying Edman degradation to the peptide;
analyzing the first portion using mass spectrometry; and
analyzing the second portion using mass spectrometry.

6. (canceled)

7. The method of claim 1, wherein at least some of the statistical distributions are Gaussian.

8. The method of claim 1, comprising analyzing the sample using MS1.

9. The method of claim 1, wherein the sample has a mass of 100 pg or less.

10. The method of claim 1, wherein the sample comprises a single cell.

11. The method of claim 1, wherein the sample comprises a regulatory molecule.

12. The method of claim 1, further comprising an internal mass standard.

13. The method of claim 4, further comprising isotopically labeling the second portion with a second isotope having a different mass than the first isotope.

14. The method of claim 4, comprising analyzing the first portion using MS1.

15. The method of claim 4, comprising analyzing the second portion using MS1.

16. The method of claim 4, wherein analyzing the first portion using mass spectrometry and analyzing the second portion using mass spectrometry comprises:
comprising combining the first and second portions into a combined portion; and
analyzing the combined portion using mass spectrometry.

17. The method of claim 1, wherein repeating the analyzing step one or more times comprises repeating the analyzing step using mass spectrometry at a different voltage.

18. The method of claim 4, further comprising isotopically labeling the second portion with a second isotope having a different mass than the first isotope.

19. The method of claim 4, wherein analyzing the first portion using mass spectrometry and
analyzing the second portion using mass spectrometry comprises:
combining the first and second portions into a combined portion; and
analyzing the combined portion using mass spectrometry.

20. The method of claim 1, wherein the sample comprises a peptide.

21-22. (canceled)

23. The method of claim 1, wherein the mass spectrometry comprises MS1

24-26. (canceled)
